(19) United States
(12) Patent Application Publication
(10) Pub. No.: US 2010/0331430 A1
(43) Pub. Date: Dec. 30, 2010

(54) PHARMACEUTICAL COMPOSITIONS CONTAINING PROPIONIC PRESERVATIVE COMPONENTS

(75) Inventor: Orest Olejnik, Coto De Caza, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/824,387

(22) Filed: Jun. 28, 2010

Related U.S. Application Data
(60) Provisional application No. 61/221,578, filed on Jun. 30, 2009.

(51) Int. Cl. A61K 47/12; A61P 27/02

(52) U.S. Cl. 514/784

(57) ABSTRACT

The invention provides ophthalmic compositions preserved using propionic preservative components alone or in combination with at least one additional preservative. In particular, an improvement in anti-microbial activity against bacteria is seen in addition to activity specific to fungal organisms and/or mold.

### CROSS-REFERENCE TO RELATED APPLICATION

This application is based, and claims priority under 35 U.S.C. §120 to U.S. Provisional Patent Application No. 61/221,578 filed on Jun. 30, 2009, and which is incorporated herein by reference.

### FIELD OF THE INVENTION

The present invention relates generally to pharmaceutical compositions. In particular, the present invention relates to ophthalmic compositions containing an active drug and at least one propionic preservative component

### BACKGROUND OF THE INVENTION

Preservatives are used in multi-use ophthalmic compositions to prevent microbial contamination of the composition after the packaging has been opened. A number of preservatives have been developed including quaternary ammonium salts such as benzalkonium chloride; mercury compounds such as phenylmercuric acetate and thimerosal; alcohols such as chlorobutanol and benzyl alcohol; and others.

Propionic acid and salts thereof are known antifungal agents having found utility as inhibitors of mold in the baking industry. However, to date these compounds have not found general application in the preservation of ophthalmic compositions.

### SUMMARY OF THE INVENTION

The invention provides ophthalmic compositions preserved using propionic preservative components alone or in combination with at least one additional preservative. In particular, an improvement in anti-microbial activity against bacteria is seen in addition to activity specific to fungal organisms and/or molds.

In one embodiment of the invention, there are provided compositions including a pharmaceutically active component effective in treating ocular disorders in a subject in need thereof; at least one propionic preservative component; and optionally, at least one additional preservative component; wherein the composition is an ophthalmic liquid.

In another embodiment of the invention, there are provided methods for preserving an ophthalmic solution. Such methods can be performed, for example, by adding to the solution at least one propionic preservative component in an amount sufficient to preserve the ophthalmic solution.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with, and the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, and formulation.

As used herein, “propionic preservative component” refers, in some embodiments of the invention, to propionic acid and pharmaceutically acceptable salts thereof. In other embodiments, the propionic preservative component is an alkali propionate or alkaline earth propionate. In certain embodiments the propionic preservative component is sodium propionate, potassium propionate, magnesium propionate, calcium propionate, and the like.

As used herein, “propionic preservative component” may also refer to polymeric propionates such as cellulose actetate propionate (CAP), acrylamidomethyl cellulose actetate propionate, and the like.

In one embodiment of the invention, the propionic preservative component is used in combination with at least one additional preservative. Without wishing to be bound by theory, it is believed that the combination of a propionic preservative component and, for example, a stabilized chlorine dioxide preservative, provides enhanced activities over and above each component alone. In particular, it is believed that the presence of a propionate moiety augments the preservative action of oxychloro complex compounds, e.g., Purite, and increases activity against fungal organisms.

In one embodiment, the use of CAP in the compositions of the invention enables greater preservative effectiveness of ophthalmic preservatives, e.g., Purite, and at more preferential physiological pH's. CAP combined with propionic acid or a pharmaceutically acceptable salt thereof further augments antimicrobial activity. In addition, the physicochemical properties of CAP provide additional drug delivery and increased pre-corneal retention.

Yet another benefit realized by the use of CAP is the increased stability of propionate salts in the presence of CAP. In other words, the use of CAP in invention ophthalmic compositions not only provides synergistic preservative activity but also improves the stability of the overall preservative system.

The term “stabilized chlorine dioxide” is well known in the industry and by those skilled in the art. The term “stabilized chlorine dioxide” as used herein means, for example, one or more chlorine dioxide-containing complexes disclosed in U.S. Pat. Nos. 4,696,811 and 4,689,215, which are incorporated herein by reference. Chlorites include metal chlorite salts, particularly alkali metal chlorites. A specific example of a chlorite salt which is useful as a chlorine dioxide precursor is sodium chlorite. Among the preferred stabilized chlorine dioxide complexes are carbonate and bicarbonate complexes. The exact chemical composition of many of these stabilized chlorine dioxide precursors is not completely understood. The manufacture or production of certain chlorine dioxide precursors is described in McNicholas U.S. Pat. No. 3,278,447, which is hereby incorporated in its entirety by reference herein.

A commercially available stabilized chlorine dioxide which can be utilized in the compositions disclosed herein is the proprietary stabilized chlorine dioxide of BioCide International, Inc. of Norman, Okla., sold under the trademark Purite®. Other suitable stabilized chlorine dioxide products include that sold under the trademark Dura Klor® by Rio Linda Chemical Company, Inc., and that sold under the trademark Antheium Dioxide® by International Dioxide, Inc. The amount of stabilized chlorine dioxide used depends upon the pharmaceutically active component, other excipients, and other aspects of the formulation process. Such a determination can readily be made by a person of ordinary skill in the art, without undue experimentation. While the amount of stabilized chlorine dioxide may vary widely, a concentration between 30 ppm and 500 ppm is useful in many compositions. In other compositions, from 50 ppm and 150 ppm stabilized chlorine dioxide is used.

As used herein, a “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted.

A liquid which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

As is known in the art, buffers are commonly used to adjust the pH to a desirable range for ophthalmic use. Generally, a pH of around 6-8 is desired, however, this may need to be adjusted due to considerations such as the stability or solubility of the pharmaceutically active component or other excipients. Many buffers including salts of inorganic acids such as phosphate, borate, and sulfate are known. Although any buffer may be used in the compositions disclosed herein, in certain situations it is particularly useful to use a borate/boric acid buffer in the compositions disclosed herein. The term “borate/boric acid buffer” refers to any combination of boric acid and one or more of the conjugate bases such that the pH is adjusted to the desired range. While not intending to limit the scope of the invention in any way, or be bound in any way by theory, it is believed that the borate/boric acid buffer may boost the antimicrobial properties of stabilized chlorine dioxide.

Another commonly used excipient in ophthalmic compositions is a viscosity-enhancing, or a thickening agent. Thickening agents are used for a variety of reasons, ranging from improving the form of the formulation for convenient administration to improving the contact with the eye to improve bioavailability. The viscosity-enhancing agent may comprise a polymer containing hydrophilic groups such as monosaccharides, polysaccharides, ethylene oxide groups, hydroxyl groups, carboxylic acids or other charged functional groups. While not intending to limit the scope of the invention, some examples of useful viscosity-enhancing agents are sodium carboxymethylcellulose, hydroxypropylmethylcellulose, povidone, polyvinyl alcohol, and polyethylene glycol.

In ophthalmic solutions, tonicity agents often are used to adjust the composition of the formulation to the desired isotonic range. Tonicity agents are well known in the art and some examples include glycerin, mannitol, sorbitol, sodium chloride, and other electrolytes.

A surfactant may be used for assisting in dissolving an excipient or an active agent, dispersing a solid or liquid in a composition, enhancing wetting, modifying drop size, or a number of other purposes. Useful surfactants, include, but are not limited to sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phosphalipids, phosphatidyl chloline, phosphatidyl serine, and the like.

Other excipient components which may be included in the ophthalmic preparations are chelating agents. A useful chelating agent is edetate disodium, although other chelating agents may also be used in place or in conjunction with it.

Pharmaceutically active components contemplated for use in the practice of the invention include, but are not limited to, prostaglandins, prostamides, retinoids, [symbol] - adrenergic agents, tyrosine kinase inhibitors, cyclosporine analogs, non-steroidal anti-inflammatory drugs, steroids, and the like.

In, one embodiment, the pharmaceutically active components include a carboxylic acid, a carboxylic acid ester, or a carboxylic acid amide. In another embodiment, the pharmaceutically active component is a prostaglandin or prostamide such as bimatoprost, latanoprost, travoprost, unoprostone isopropyl, and the like, which have carboxylic acid, ester, or amide groups. In another embodiment, the pharmaceutically active component comprises a sulfur atom. Other functional groups that may be susceptible to stabilized chlorine dioxide are amines, phenols, alcohols, aromatic amino acids, non-conjugated double bonds, and similar groups. While not intending to be limiting, or to be bound by theory, non-active excipients comprising one or more of the aforementioned functional groups should be stabilized by citric acid such that they can be used with stabilized chlorine dioxide.

Ocular disorders that can be effectively treated by invention compositions include, but are not limited to, dry eye, glaucoma, inflammation, keratitis, conjunctivitis, ocular infections, or ocular allergies.

In another embodiment of the invention, there are provided methods for preserving an ophthalmic solution comprising adding to the solution at least one propionic preservative component in an amount sufficient to preserve the ophthalmic solution.

### EXAMPLES

A preservative effectiveness study was performed to determine if adding cellulose acetate increased the efficacy of the purite preservative in Optive. For this study, two different concentrations of cellulose acetate (0.1% and 0.5%) were added to Optive samples and analyzed against Optive alone.

A standard APET test USP <51>/EP 5.1.3 was performed comparing the three different samples using the standard five organisms defined in the pharmacopeias. For the three bacteria

(S. aureus, P. aeruginosa, and E. coli) at 6 hour time point, there was a greater log reduction with the addition of 0.5% cellulose acetate that was between 0.80 to 1.3 log greater log reduction than Optive alone. (Refer to attached Table). All time points met the criteria as stated in USP and EU pharmacopoeias.

For the yeast and mold organisms, at the day 14 test point, the 0.5% cellulose acetate also showed a 1.55 greater log reduction for C. albicans and a 0.48 greater log reduction for A. brasiliensis than Optive alone. All time points met the criteria as stated in USP and EU pharmkopoeias for ophthalmic preparations.

| | Time Point | | | | | | |
|---|---|---|---|---|---|---|---|
| | Date: 7 Apr. 2010 | Date: 7 Apr. 2010 | | Date: 8 Apr. 2010 | | | |
| | 14 Apr. 2010 | Time: 1545 | | Time: 1545 | | Date: 14 Apr. 2010 | |
| | Time: 0945 | 6 HR | | 24 HR | | 7 DAY | |
| | 0 HR | Average | Log | Average | Log | Average | Log |
| | Control Count | Count | reduc- | Count | reduc- | Count | reduc- |
| Organism/Product | CFU | CFU/ml. | tion | CFU/ml. | tion | CFU/ml. | tion |
| S. aureus | 1.5 × 10⁷ | 3.2 × 10⁴ | 2.67 | <10 | 6.18 | <10 | 6.18 |
| Optive | | | | | | | |
| S. aureus | | 4.2 × 10⁴ | 2.55 | <10 | 6.18 | <10 | 6.18 |
| Optive + 0.1% cell. | | | | | | | |
| acetate | | | | | | | |
| S. aureus | | 1.6 × 10³ | 3.97 | <10 | 6.18 | <10 | 6.18 |
| Optive + 0.5% cell. | | | | | | | |
| acetate | | | | | | | |
| P. aeruginosa | 1.8 × 10⁶ | 7.7 × 10³ | 2.37 | <10 | 5.26 | <10 | 5.26 |
| Optive | | | | | | | |
| P. aeruginosa | | 1.2 × 10³ | 3.18 | <10 | 5.26 | <10 | 5.26 |
| Optive + 0.1% cell. | | | | | | | |
| aectate | | | | | | | |
| P. aeruginosa | | 1.5 × 10³ | 3.20 | <10 | 5.26 | <10 | 5.26 |
| Optive + 0.5% cell. | | | | | | | |
| acetate | | | | | | | |
| E. coli | 2.2 × 10⁶ | 3.1 × 10² | 2.85 | <10 | 5.34 | <10 | 5.34 |
| Optive | | | | | | | |
| E. coli | | 1.4 × 10³ | 3.2 | <10 | 5.34 | <10 | 5.34 |
| Optive + 0.1% cell. | | | | | | | |
| acetate | | | | | | | |
| E. coli | | 4.9 × 10² | 3.65 | <10 | 5.34 | <10 | 5.34 |
| Optive + 0.5% cell. | | | | | | | |
| acetate | | | | | | | |
| C. albicans | 1.6 × 10⁶ | | | | | 3.6 × 10² | 3.65 |
| Optive | | | | | | | |
| C. albicans | | | | | | 2.3 × 10¹ | 4.84 |
| Optive + 0.1% cell. | | | | | | | |
| acetate | | | | | | | |
| C. albicans | | | | | | <10 | 5.20 |
| Optive + 0.5% cell. | | | | | | | |
| acetate | | | | | | | |
| A. brasiliensis | 2.1 × 10⁵ | | | | | 1.2 × 10³ | 2.24 |
| Optive | | | | | | | |
| A. brasiliensis | | | | | | 8 × 10² | 2.42 |
| Optive + 0.1% cell. | | | | | | | |
| acetate | | | | | | | |
| A. brasiliensis | | | | | | 4 × 10² | 2.72 |
| Optive + 0.5% cell. | | | | | | | |
| acetate | | | | | | | |

| Time Point | | |
|---|---|---|
| Date: 21 Apr. 2010 | Date: 28 Apr. 2010 | Date: 5 May 2010 |
| 14 DAY | 21 DAY | 28 DAY |

| | Average | Log | Average | Log | Average | Log |
|---|---|---|---|---|---|---|
| | Count | reduc- | Count | reduc- | Count | reduc- |
| Organism/Product | CFU/ml. | tion | CFU/ml. | tion | CFU/ml. | tion |
| S. aureus | <10 | 6.18 | <10 | 6.18 | <10 | 6.18 |
| Optive | | | | | | |
| S. aureus | <10 | 6.18 | <10 | 6.18 | <10 | 6.18 |
| Optive + 0.1% cell. | | | | | | |
| acetate | | | | | | |
| S. aureus | <10 | 6.18 | <10 | 6.18 | <10 | 6.18 |
| Optive + 0.5% cell. | | | | | | |
| acetate | | | | | | |
| P. aeruginosa | <10 | 5.26 | <10 | 5.26 | <10 | 5.26 |
| Optive | | | | | | |
| P. aeruginosa | <10 | 5.26 | <10 | 5.26 | <10 | 5.26 |
| Optive + 0.1% cell. | | | | | | |
| aectate | | | | | | |
| P. aeruginosa | <10 | 5.26 | <10 | 5.26 | <10 | 5.26 |
| Optive + 0.5% cell. | | | | | | |
| acetate | | | | | | |
| E. coli | <10 | 5.34 | <10 | 5.34 | <10 | 5.34 |
| Optive | | | | | | |
| E. coli | <10 | 5.34 | <10 | 5.34 | <10 | 5.34 |
| Optive + 0.1% cell. | | | | | | |
| acetate | | | | | | |
| E. coli | <10 | 5.34 | <10 | 5.34 | <10 | 5.34 |
| Optive + 0.5% cell. | | | | | | |
| acetate | | | | | | |
| C. albicans | <10 | 5.20 | <10 | 5.20 | <10 | 5.20 |
| Optive | | | | | | |
| C. albicans | <10 | 5.20 | <10 | 5.20 | <10 | 5.20 |
| Optive + 0.1% cell. | | | | | | |
| acetate | | | | | | |
| C. albicans | <10 | 5.20 | <10 | 5.20 | <10 | 5.20 |
| Optive + 0.5% cell. | | | | | | |
| acetate | | | | | | |
| A. brasiliensis | 2.1 × 10³ | 2.00 | 1.1 × 10³ | 2.28 | 3.3 × 10² | 2.80 |
| Optive | | | | | | |
| A. brasiliensis | 1.5 × 10³ | 2.15 | 9.7 × 10² | 2.34 | 6 × 10² | 2.54 |
| Optive + 0.1% cell. | | | | | | |
| acetate | | | | | | |
| A. brasiliensis | 1.5 × 10³ | 2.15 | 6.7 × 10² | 2.50 | 6.4 × 10² | 2.52 |
| Optive + 0.5% cell. | | | | | | |
| acetate | | | | | | |

PAM APET study: Optive challenge with cellulose acetate

### Preservative Efficacy Acceptance Criteria

European Pharmacopoeia Acceptance Criteria

| | | Log Reduction | | | | | |
|---|---|---|---|---|---|---|---|
| | Type | 6 hr | 24 hr | 2 day | 7 day | 14 day | 28 day |
| | Bacteria | | | | | | |
| Parenteral | A criteria | 2 | 3 | — | — | — | NR |
| and | B criteria | — | 1 | — | 3 | — | NI |
| Ophthalmic | | | | | | | |
| | Fungi | | | | | | |
| | A criteria | — | — | — | 2 | — | NI |
| | B criteria | — | — | — | — | 1 | NI |

NR—No Recovery
NI—No increase

| | |
|---|---|
| USP | Not less than 1.0 log reduction from the initial calculated count |
| criteria | at 7 days, not less than 3.0 log reduction from the initial count |
| | at 14 days and no increase from the 14 days count at 28 days |

What is claimed is:

1. A composition comprising
a pharmaceutically active component effective in treating ocular disorders in a subject in need thereof;
at least one propionic preservative component; and
optionally, at least one additional preservative component;
wherein the composition is an ophthalmic liquid.

2. The composition of claim 1, wherein the propionic preservative component is propionic acid or a pharmaceutically acceptable salt thereof.

3. The composition of claim 2, wherein the pharmaceutically acceptable salt is an alkali propionate or alkaline earth propionate.

4. The composition of claim 3, wherein the pharmaceutically acceptable salt is sodium propionate, potassium propionate, calcium propionate, or magnesium propionate.

5. The composition of claim 1, wherein the propionic preservative component is a polymeric propionate.

6. The composition of claim 5, wherein the polymeric propionate is cellulose acetate propionate or acrylamidomethyl cellulose acetate propionate.

7. The composition of claim 1, wherein the additional preservative component is a chlorine dioxide precursor.

8. The composition of claim 7, wherein the chlorine dioxide precursor is stabilized chlorine dioxide (SCD), alkali metal chlorites, alkaline earth metal chlorites, chlorine dioxide-containing complexes such as complexes of chlorine dioxide with carbonate, chlorine dioxide with bicarbonate, and mixtures thereof.

9. The composition of claim 1 wherein the propionic preservative component is present in the composition in an amount from 0.1 wt % to 5 wt %.

10. The composition of claim 1 wherein the additional preservative component is present in amount from 0.001 wt % to 0.050 wt %.

11. The composition of claim 1 wherein the pharmaceutically active component is a prostaglandin, prostamide, retinoid, α-adrenergic agent, tyrosine kinase inhibitor, cyclosporine analogs, non-steroidal anti-inflammatory drugs, or steroid.

12. The composition of claim 1 wherein the ocular disorder is dry eye, glaucoma, inflammation, keratitis, conjunctivitis, ocular infections, or ocular allergies.

13. A method for preserving an ophthalmic solution comprising adding to the solution at least one propionic preservative component in an amount sufficient to preserve the ophthalmic solution.

14. The method of claim 13, wherein the propionic preservative component is propionic acid or a pharmaceutically acceptable salt thereof.

15. The method of claim 13, wherein the pharmaceutically acceptable salt is sodium propionate, calcium propionate, potassium propionate, or magnesium propionate.

16. The method of claim 13, wherein the propionic preservative component is a polymeric propionate.

17. The method of claim 13, wherein the polymeric propionate is cellulose acetate propionate or acrylamidomethyl cellulose acetate propionate.

18. The method of claim 13, wherein the ophthalmic solution is effective in treating dry eye, glaucoma, inflammation, keratitis, conjunctivitis, ocular infections, or ocular allergies.
